(19) United States
(12) Patent Application Publication
(10) Pub. No.: US 2011/0112390 A1
(43) Pub. Date: May 12, 2011

(54) APPARATUS AND METHOD FOR BLOOD CLOTTING DIAGNOSTICS

(76) Inventor: Rolf Zander, Mainz (DE)

(21) Appl. No.: 12/991,388

(22) PCT Filed: Apr. 28, 2009
(86) PCT No.: PCT/EP09/55145

§ 371 Date: Jan. 24, 2011

(30) Foreign Application Priority Data
May 8, 2008 (DE) 10 2008 022 884.2

(51) Int. Cl. A61B 5/00

(52) U.S. Cl. 600/369

(57) ABSTRACT

In order to be able still more precisely to determine the blood clotting activity in a patient according to the invention there is provided a method of extracorporeal determination of the blood clotting activity of a blood sample of an individual, in which the clotting time which elapses until the blood sample reaches a given degree of clotting or the degree of clotting reached by the blood sample within a given time is measured or in which in a corresponding manner the fibrinolysis time or the degree of fibrinolysis of a blood sample which has previously clotted to a certain degree is measured, the method being characterised in that measurement of the clotting/fibrinolysis time or the degree of clotting/fibrinolysis is effected with the exclusion of air. In addition according to the invention there is proposed a corresponding apparatus for the extracorporeal determination of the blood clotting activity wherein the apparatus has a measurement chamber for measuring the clotting time, the degree of clotting, the fibrinolysis time or the degree of fibrinolysis of a blood sample, the apparatus being characterised in that the measurement chamber is of such a configuration that measurement of the clotting/fibrinolysis time or the degree of clotting/fibrinolysis is effected with the exclusion of air.

The present invention concerns a method of extracorporeal determination of the blood clotting activity of a blood sample of an individual, in which the clotting time which elapses until the blood sample reaches a given degree of clotting or the degree of clotting reached by the blood sample within a given time is measured. In addition in accordance with that method extracorporeal determination of the blood clotting activity of a blood sample of an individual can also be effected in that in a corresponding fashion the fibrinolysis time which elapses until a blood sample clotted to a certain degree again reaches a given degree of fibrinolysis or the degree of fibrinolysis which a blood sample which has clotted to a certain degree reaches within a given time is measured. In addition the present invention also concerns an apparatus for extracorporeal determination of the blood clotting activity of a blood sample of an individual, wherein the apparatus has a measuring chamber for measuring the clotting time or the degree of clotting, or the fibrinolysis time or the degree of fibrinolysis.

The present invention is in the field of clotting diagnostics. In the context of this application the term blood clotting is used to denote the interplay of the physiological processes leading to clotting of the blood, that is to say the formation of a blood clot. Blood clotting (haemostasis) is generally sub-divided into cellular haemostasis and plasmatic haemostasis. Cellular haemostasis includes adhesion and aggregation of thrombocytes and the activation of additional thrombocytes, in which case a so-called “white thrombocyte thrombus” is formed. In contrast, during plasmatic haemostasis, constituents of the blood plasma form a mesh of fibrin threads and thus the so-called “red thrombus”, wherein the red colouration is caused by bound-in erythrocytes. Plasmatic haemostasis is triggered and regulated by way of the so-called clotting cascade in which a series of different clotting factors are involved. The aforementioned processes are physiologically and functionally very closely linked together and are here summarised by the overall term of blood clotting.

Fibrinolysis is the physiological breakdown of a blood clot. In that case the enzyme plasmin cuts the fibrin polymers which have been produced in blood clotting into different decomposition products which are transported away by way of the blood. Ultimately that results in breakdown of a blood clot. Fibrinolysis is already activated with the onset of blood clotting and serves by virtue of the fact that it has an antagonistic action in relation to blood clotting for regulation of blood clotting activity. Therefore the term blood clotting activity in relation to the present invention embraces not only the aspect of haemostasis (blood clotting) but also fibrinolysis (fibrin cleaving).

A method of determining the blood clotting activity is thrombelastography. With that method it is possible to investigate both the aspect of blood clotting and also that of fibrinolysis. Further methods of determining blood clotting activity are determining the thromboplastin time (TPT), activated partial thromboplastin time (aPTT), the thrombin time (TT), the prothrombin time, ecarin clotting time (ECT) and activated clotting time (ACT) as well as impedance aggregometry or the measurement of thrombocytes adhesion and aggregation.

The present invention is aimed in particular at improving patient-close immediate diagnostics (point of care diagnostics or POC) of blood clotting. Point of care diagnostics has inter alia the advantage that the materials to be investigated in the context of the diagnostics procedure, for example samples taken from the body of the individual to be investigated, can be investigated in respect of time more or less immediately after the samples are taken. It is particularly advantageous in that respect that, when performing the diagnostics, further aspects can also be taken into consideration such as for example physiological parameters of the individual, which are ascertained shortly before, during or after the step of taking the sample.

A human patient with a body temperature of 32° C. is in the condition of hypothermia in comparison with a human being at a normal body temperature of about 37° C. In the hypothermia condition the human blood clotting function is considerably limited. If clotting diagnostics is effected for a hypothermal patient, which is carried out at a temperature above the currently prevailing body temperature of that patient, that would lead to an incorrect diagnosis as the blood sample of the hypothermal patient, if it is returned to the level of the normal body temperature during the diagnostic procedure (as is usual with many devices), would fake a normal clotting status which does not occur at all in the actually colder blood of the patient. Such a misdiagnosis is therefore usually precluded by the body temperature of the patient being determined immediately before, during or after the blood sampling operation and subsequent determination of the blood clotting activity of the blood sample taken from that patient is effected at a temperature which is set to a given body temperature.

As was already mentioned in the opening part of this specification point of care diagnostics affords substantial advantages over diagnostics at a later moment in time as for example in a central laboratory. The aim of the present invention is to still further reinforce those advantages in order to be able still more precisely to determine the blood clotting activity occurring in a patient at a given moment in time.

Therefore the object of the present invention is to provide a correspondingly improved method with which blood clotting activity which the blood in a patient has can be still more accurately analysed. Another object of the invention is also to provide an apparatus with which such a method can be carried out.

According to the invention that object is attained in that, in the method set forth in the opening part of this specification for extracorporeal determination of the blood clotting activity of a blood sample of an individual, measurement of the clotting time or the degree of clotting, or the fibrinolysis time or the degree of fibrinolysis, is carried out with the exclusion of air.

In connection with the present invention the blood clotting time is taken to mean the time which elapses until a blood sample reaches a given degree of clotting. In connection with the present invention the degree of clotting of a blood sample is taken to denote the extent to which a blood clot is formed in the blood sample to be investigated, within a given period of time. In connection with the present invention the fibrinolysis time is taken to mean the time which elapses until a blood clot formed in a blood sample which has clotted to a certain degree breaks up again to a certain degree within a given time. In connection with the present invention the degree of fibrinolysis of a blood sample is used to denote the extent to which a blood clot which has formed in a blood sample which has clotted to a certain degree breaks up again within a given time.

Carrying out the measurement with the exclusion of air means that the blood sample does not come into contact with air at least during measurement of the blood clotting time or the degree of blood clotting or the fibrinolysis time or the degree of fibrinolysis. In other words that means that the blood sample, at least during measurement of the above-mentioned parameters, is in an environment in which no air is present and into which also no air can penetrate at least during the measurement time.

Here the term “air” or “ambient air” is used to mean the gas mixture which surrounds a human being in his everyday life. Accordingly “air” or “ambient air” in accordance with the present invention is composed primarily of the gases nitrogen (about 78.1% by volume; partial pressure=594 mmHg) and oxygen (about 20.9% by volume; partial pressure 159 mmHg). In addition the “air” or “ambient air” also has a proportion of about 0.9% by volume of argon (partial pressure 6.8 mmHg) and a proportion of about 0.03% by volume of carbon dioxide (partial pressure 0.2 mmHg) as well as traces of hydrogen. In addition the “natural atmosphere” can have a proportion of water vapour, which proportion can be in the range of 0 to 6.2% by volume (partial pressure 0 to 47 mmHg) and can vary greatly depending on respective water vapour saturation and temperature. The respective proportion of the gases in the air which are not water vapour is reduced in accordance with the proportion of water vapour in the air. The details of the partial pressures of the gases relate to barometer pressure of 760 mmHg.

The invention is based on the concept that many physiological processes are pH-dependent, that is to say they take place in the optimum fashion in an optimum pH range and outside that range take place in sub-optimal fashion or cannot take place at all. It has been found that blood clotting also depends in a certain way on the pH-value of the medium in which blood clotting takes place. For example it was possible to show that many blood clotting factors have an activity dependent on the pH-value. Thus the activity of individual clotting factors can be reduced by about half upon a reduction in the pH-value from 7.40 to 7.20 (corresponding to a base deficit BE of −12.5 mmol/l), while it can double with an increase in the pH-value from 7.40 to 7.60 (corresponding to a base excess BE of +16.5 mmol/l). Accordingly it is of essential importance that, when determining the blood clotting activity of a blood sample, the pH-value of the blood sample is kept at a defined pH-level from being taken from the patient to the end of measurement of the blood clotting activity. Preferably that pH-value corresponds to the pH-value which actually prevails in the blood of the patient.

The present invention takes account of that concept in that measurement of the blood clotting activity is effected with the exclusion of air. That has the advantage that no gas exchange can take place between the blood sample and the ambient air at least during the measurement operation. The concentration of given gases in the blood has an influence on the pH-value of the blood. Thus the rise in the concentration of CO₂ dissolved in the blood leads to a reduction in the pH-value while a reduction in the CO₂ concentration leads to the blood becoming more alkaline.

If a blood or plasma sample is handled in such a way that the sample can come into contact with air there is the danger that CO₂ can diffuse out, which leads to a reduction in the CO₂ concentration in the blood. The reduction in the CO₂ partial pressure (pCO₂) from the originally normal 40 mmHg (arterial blood) or about 50 mmHg (venous blood) to values therebelow leads to alkalinisation of the sample with a rise in the pH-value to above the normal value of about 7.40. Measurement of the blood clotting activity of a blood sample which as a consequence of coming into contact with air has a correspondingly increased pH-value would therefore lead to a result which does not exactly reflect the blood clotting activity actually prevailing in the blood of the patient, because of the above-discussed pH-dependency of given blood clotting factors. According to the invention that measurement error is avoided by measurement of the blood clotting activity of the blood sample being effected with the exclusion of air.

In a preferred embodiment of the present invention the method of determining the blood clotting activity is additionally characterised in that the blood sample is also taken from the body of the individual with the exclusion of air. A further preferred method according to the present invention is characterised in that the blood sample is also kept in store until the measurement operation, with the exclusion of air. In a further preferred embodiment of the method according to the invention transfer of the blood sample into an apparatus for measuring the blood clotting activity is also effected with the exclusion of air. In a particularly preferred method both the step of taking the sample and also storing it and also transferring it into a measuring apparatus are effected with the exclusion of air. That provides that the blood sample comes into contact with air as little as possible down to substantially not at all, whereby the risk of the exchange of gases between the blood sample and the ambient air can be excluded for a large part to almost completely, which leads to still more precise and still more authentic results.

In an alternative embodiment of the invention measurement of the blood activity is effected without the blood sample coming into contact with a gas phase during the measurement operation. That can be achieved for example by measurement of the blood clotting activity being effected in a measuring apparatus in which the blood sample is disposed during the measurement operation in a space in which there is neither air nor another gas. That can be achieved for example by that space, for example the measurement chamber provided in the measurement apparatus, being completely filled with the blood sample and then air-tightly closed off. In a preferred embodiment the step of taking the blood sample and/or storage of the blood sample as far as the measurement operation and/or transfer of the blood sample into the measurement apparatus are effected without the blood sample coming into contact with a gas phase.

In an alternative embodiment of the invention the blood sample which is arranged in the measurement chamber is overlaid with a gas barrier fluid so that the blood sample cannot come into contact with a gas phase present above that gas barrier fluid. A suitable gas barrier fluid is any fluid which is characterised in that in comparison with blood it is of higher viscosity and lower density and can form a layer on the blood sample without mixing therewith. In addition a gas barrier fluid according to the invention is distinguished in that it absorbs substantially no CO₂ and/or no O₂ and/or no water vapour or allows same to diffuse therethrough. Such fluids can be produced on the basis of paraffin or silicone oils or ethylene glycol.

As an alternative it is also possible to use barrier gases which are markedly heavier (high density) than air and lighter (lower density) than blood to overlay the blood sample. A barrier gas which is for example suitable for those purposes is sulphur hexafluoride (SF₆).

Because the gas barrier fluid allows substantially no diffusion of gases there can also be no gas exchange between the blood sample and the gas phase above the gas barrier fluid so that measurement of the blood clotting activity can be effected with the exclusion of air.

In a further alternative embodiment measurement of the blood clotting activity in accordance with this invention is carried out under an artificial atmosphere. Here the term “artificial atmosphere” is used to denote basically any gas or gas mixture which differs in its nature and composition from the ambient air (“natural atmosphere”). The artificial atmosphere is preferably selected from a gas having a predetermined CO₂ partial pressure, O₂ partial pressure and/or H₂O partial pressure that differs from the natural ambient air, or a protective gas selected from nitrogen gas and/or an inert gas.

The partial pressure is generally used to denote the pressure which in a gas mixture can be associated with a given gas contained therein. In that respect the partial pressure corresponds to the overall pressure that the component would exert if solely filling the overall volume. In accordance with the present invention the partial pressure of a gas dissolved in a fluid is used to denote the pressure which corresponds to the partial pressure of the corresponding gas in a gas phase which is in contact with the fluid, which would lead to the corresponding gas being in a diffusion equilibrium at the interface of the gas and the fluid.

That means that whenever reference is made in the context of this application to the partial pressure of a gas in a fluid, the second definition of partial pressure is to be applied. In contrast whenever reference is made to the partial pressure of a gas in a gas mixture, the first broad definition of partial pressure is to be applied.

A preferred method of the present invention is characterised in that a blood pre-sample is taken or a blood sample aliquot is branched off the blood sample immediately before, after or during the operation of taking the blood sample intended for determining the blood clotting activity. One or more of the following parameters are then determined for that blood pre-sample or blood sample aliquot: CO₂ partial pressure, O₂ partial pressure and pH-value. That procedure has the advantage that subsequently in connection with determining the blood clotting activity of the blood sample it is possible to check the extent to which the blood sample has the same or a differing CO₂ partial pressure, O₂ partial pressure or pH-value during the operation of determining the blood clotting activity. That is to say it is possible in that way to check whether the step of determining the blood clotting activity is effected under physiological or pathological conditions, that is to say under the conditions which prevailed in the patient when the blood sample was taken.

In a preferred embodiment of the invention in which measurement of the blood clotting activity is effected under an artificial atmosphere, the predetermined CO₂ partial pressure of the artificial atmosphere corresponds to the CO₂ partial pressure of the blood pre-sample or the blood sample aliquot. In a further preferred embodiment the predetermined O₂ partial pressure of the artificial atmosphere corresponds to the O₂ partial pressure of the blood pre-sample or the blood sample aliquot. In a further preferred embodiment both the CO₂ partial pressure and also the O₂ partial pressure of the artificial atmosphere correspond to the partial pressures of the corresponding gases in the blood pre-sample or the blood sample aliquot. In still a preferred embodiment of the invention the predetermined H₂O partial pressure of the artificial atmosphere corresponds to the H₂O partial pressure which is reached in that artificial atmosphere when there is water vapour saturation of the artificial atmosphere at the temperature at which blood clotting activity measurement is performed. That prevents the blood sample being concentrated by evaporation of water out of the blood sample, whereby the pH-value would normally fall. The desired H₂O partial pressure of the artificial atmosphere can be achieved for example by the artificial atmosphere, before it comes into contact with the blood sample, being passed through a water-filled fit which is set to the temperature at which blood clotting activity measurement is also performed.

Preferably blood clotting activity measurement is effected in accordance with the method of the invention at a given temperature. Preferably that temperature is the body temperature which was measured shortly before, after or during the operation of taking the blood sample from the patient.

In a special standardised embodiment of the method according to the invention blood clotting activity measurement is effected at 37° C. In a further special standardised embodiment the predetermined CO₂ partial pressure of the artificial atmosphere is 40 ±2.5 mmHg. In a further special standardised embodiment of the invention the predetermined O₂ partial pressure of the artificial atmosphere is 90 ±2.5 mmHg. The predetermined H₂O partial pressure of the artificial atmosphere in a special standardised embodiment of the invention is 47 ±2.5 mmHg.

In a preferred embodiment of the invention the gas mixture carbogen is used as the artificial atmosphere. Carbogen is a gas mixture of 5% by volume of CO₂ (partial pressure 38 mmHg) and 95% by volume of O₂ (partial pressure 722 mmHg). The advantage of using carbogen is that it has a mixture of gases which is well suited to the blood clotting diagnostics according to the invention and can be obtained at relatively low cost.

Preferably the flow of the artificial atmosphere is suitably adjusted. Preferably the flow is kept as low as possible. Particularly preferably the gas flow per minute is twice as great as the volume which the measurement chamber and the inlets and outlets connected to the measurement chamber include. Particularly preferably the gas flow is 5 ml/min. In an alternative embodiment, before the blood sample has been introduced into the measurement apparatus, the measurement apparatus is thoroughly flushed with the artificial atmosphere to ensure that there is no air in the apparatus and to equilibrate the internal surfaces of the apparatus with the artificial atmosphere. Preferably the flushing operation involves flushing once with at least three times, preferably five times and particularly preferably ten times the internal volume of the measurement chamber and the measurement chamber and/or the inlets/outlets are then fixedly closed so that no ambient air can penetrate from the exterior into the measurement chamber. Desirably a corresponding procedure is adopted after the blood sample has been introduced into the measurement chamber in order to remove any ambient air which has possibly entered when the blood sample was introduced by flushing of the gas space remaining after the blood sample is introduced. Preferably in this case also at least three times and preferably five times and particularly preferably ten times the volume of the remaining gas space is used.

In an alternative embodiment of the present invention a predetermined pH-value of the blood sample is maintained throughout the entire blood clotting activity measurement (clotting/fibrinolysis time or clotting/fibrinolysis degree). Preferably that pH-value to be maintained is the pH-value which was determined for a blood pre-sample taken immediately before, after or during the step of taking the blood sample intended for determining the blood clotting activity, or for a blood sample aliquot derived from the blood sample. If reagents are to be used to temporarily block or start specific steps in the clotting cascade (citrate, Ca²⁺ ions) they are to be dosed as low as possible in respect of their volume (dilution of the sample) and are to be so selected in respect of their composition (unbuffered) that their dosing does not change the original pH-value of the sample.

To maintain the original pH-value of the blood sample various points are to be observed. Thus an embodiment of the invention involves absolutely avoiding the sample being excessively centrifuged, whereby gases dissolved in the blood sample can be displaced out of the solution and can outgas. Particularly upon outgassing of CO₂, that involves a change, namely a rise in the pH-value in the blood sample, which here is precisely to be avoided. That also applies in regard to agitation of the blood sample. A preferred embodiment of the invention therefore involves absolutely avoiding agitating the blood sample prior to or during blood clotting activity measurement so greatly that gases can increasingly issue from the blood sample. Preferably in the method according to the invention therefore the blood sample is not centrifuged or agitated at all or it is only so slowly centrifuged or agitated that there is no expectation of increased outgassing.

In a preferred embodiment of the invention no reagents are added to the blood sample before or during the blood clotting activity measurement. In an alternative embodiment as few reagents as possible are added to the blood sample. Those reagents can be selected for example from a citrate solution and a Ca²⁺ solution. The obligatory addition of unbuffered trisodium citrate solution for clotting inhibition of the blood sample and the later addition of unbuffered calcium solution for starting the clotting cascade is to be so selected that the respective concentrations are kept as high as possible and thus the necessary volumes are kept as low as possible to prevent dilution-induced pH-drop.

In a further preferred embodiment of the invention the addition of possibly required additives is effected pH-neutral, that is to say the substances are either so selected that they have no influence on the pH-value of the blood sample (substances which react neither acid nor alkaline in aqueous medium) or in relation to a number of substances they are so selected that the influence of the individual substances on the pH-value of the blood sample is neutralised. Preferably all added reagents are unbuffered in order not to influence the original pH-value of the sample by buffer substances.

In an embodiment it is preferred that the volume of reagents which are possibly to be supplied is kept as low as possible in relation to the blood sample. If for example blood samples are diluted with plasma expanders that leads to a considerable drop in the pH-value (dilution acidosis). Dilution of the blood in vitro of 1:2 (1 part blood+1 part dilution fluid) already reduces the pH-value from 7.40 to 7.10. Preferably the volume of the total of the added reagents includes at most 5% of the volume of the blood sample. Still more preferably the volume of the total of the added reagents comprises at most 1% of the volume of the blood sample.

In addition to the above-described method according to the invention for the extracorporeal determination of the blood clotting activity of a blood sample of an individual the present invention also includes an apparatus with which extracorporeal determination of the blood clotting activity of a blood sample of an individual can be carried out. That apparatus includes a measurement chamber for measuring the clotting time which elapses until the blood sample reaches a given degree of clotting, or for measuring the degree of clotting that the blood sample reaches within a given time. So-to-speak the fibrinolysis time or the degree of fibrinolysis of a blood sample previously clotted can also be measured with the measurement chamber. According to the invention the measurement chamber is of such a configuration that the measurement of the clotting/fibrinolysis time or the clotting/fibrinolysis degree is effected with the exclusion of air. Accordingly the apparatus according to the invention is suitable for carrying out the above-described method according to the invention or an embodiment of that method. The advantages of measuring blood clotting activity with the exclusion of air have already been discussed hereinbefore.

In another embodiment of the apparatus according to the invention the measurement chamber is of such a configuration that measurement is effected without contact of the blood sample with a gas phase. Preferably feed lines which are possibly provided and by way of which a blood sample or, if required, reagents, can be added, are of such a configuration that, during the feed of the blood sample or the reagents, there is no contact between the blood sample and a gas phase or no gas phase can pass into the measurement chamber by way of that feed line.

In an alternative embodiment of the apparatus according to the invention the measurement chamber is of such a configuration that measurement of the blood clotting activity can be effected in an artificial atmosphere. Preferably the measurement chamber of the apparatus according to the invention, for those purposes, has means for the control inflow of a gas phase into the measurement chamber and/or means for the controlled discharge of a gas phase from the measurement chamber.

In an embodiment of the apparatus according to the invention it is characterised in that it has means for determining parameters of the blood sample disposed in the measurement chamber, those parameters being selected from temperature, pH-value, CO₂ partial pressure and/or O₂ partial pressure. A further embodiment of the invention has means by way of which saturation of the gas phase with water vapour can be effected, wherein those means preferably already saturate the gas phase with water vapour prior to passing into the measurement chamber. In still a further embodiment of the invention the apparatus has means for adjusting the temperature of the gas phase. In still a further embodiment of the invention the apparatus has means for adjusting the temperature of the blood sample in the measurement chamber. Preferably that involves a temperature-controllable measurement chamber, wherein the entire measurement chamber can be adjusted to the desired temperature.

In an embodiment of the invention the internal surfaces which come into contact with the blood sample, in particular the measurement chamber and the feed lines which are possibly provided and by way of which the blood sample can be introduced into the measurement chamber have low gas absorption properties to prevent equilibration of the gases carbon dioxide and oxygen of the blood sample being effected, with a surface layer. On the other hand, in certain embodiments, at least the internal surfaces of the measurement chamber are to be of such a configuration that fibrin threads can adhere to those surfaces.

In a preferred embodiment of the apparatus according to the invention the measurement chamber is cylindrical.

In a preferred embodiment of the invention, for example a cylindrical measurement chamber having a rotating or oscillating ram, the measurement chamber is of such a configuration that, in the case of a measurement chamber completely filled during the measurement operation, of a volume of between 100 and 500 μl, the ratio of the interface between the blood sample and the surface of the measurement chamber relative to the volume of the blood sample is as small as possible. Preferably the surface-volume ratio in the case of a measurement chamber volume and thus a sample volume of 100 to 500 μl is in the range of 10:1 cm⁻¹ to 25:1 cm⁻¹. A surface-volume ratio of 10:1 cm⁻¹ occurs for example when the surface is 5 cm² and the blood volume is 0.5 cm³ (500 μl), while such a ratio of 25:1 cm⁻¹ occurs with a surface of 2.5 cm² and a blood volume of 0.1 cm³ (100 μl).

In the cases in which a gas phase is present in the measurement chamber during the measurement operation the ratio of the interface between the blood sample and the gas phase with respect to the volume of the blood sample disposed in the measurement chamber is additionally to be such that the surface-volume ratio is also as low as possible. That is the case when a value of 1:1 cm⁻¹ or less is achieved.

In an embodiment of the invention the apparatus according to the invention has means for the controlled feed of reagents. An example of a reagent which can be fed by way of that apparatus is a Ca²⁺ solution.

The apparatus according to the invention and the method according to the invention are not only suitable for determining the blood clotting activity of a full-blood sample but they can also be applied for determining the blood clotting activity (clotting/fibrinolysis time or degree of clotting/fibrinolysis) of a blood derivative obtained from a blood sample (such as for example plasma).

The method and the apparatus according to the invention can basically be used in relation to any method of determining blood clotting activity. Preferably they are used for determining the clotting time, in which case the clotting time is preferably selected from the thromboplastin time (TPT), the activated partial thromboplastin time (aPTT), the thrombin time (TT), the prothrombin time, the ecarin clotting time (ECT) and the activated clotting time (ACT).

In addition the method or the apparatus according to the invention are preferably used in thrombelastography, in impedance aggregometry or in the measurement of thrombocytes adhesion and aggregation.

In thrombelastography viscoelastic changes in the full blood are measured during clot formation or clot breakdown. For that purpose the blood sample is introduced into a temperature-controlled cuvette. That cuvette rotates or oscillates against a ram which is suspended in the sample. While the blood sample gradually clots, a fibrin clot is successively formed between the inside wall of the cuvette and the ram, by way of which clot, after a certain clot stability has been reached, the rotary or oscillatory movement of the cuvette is transmitted to the ram. That movement is registered and recorded in the form of a so-called thrombelastogram. In accordance with that method it is possible on the one hand to determine the clotting time which elapses until the blood sample has reached a certain degree of clotting and on the other hand the degree of clotting that the blood sample reaches within a given time. In addition that method can also be used to determine the fibrinolysis time which elapses until the blood clot previously formed to a certain degree breaks up again to a certain degree or the degree of fibrinolysis that the blood sample which was previously clotted to a certain degree reaches again after a given time.

For the purposes of the original disclosure it is pointed out that all features as can be seen by a man skilled in the art from the present description, the drawings and the claims, even if they are described in specific terms only in connection with certain other features, can be combined both individually and also in any combinations with others of the features or groups of features disclosed here insofar as that has not been expressly excluded or technical aspects make such combinations impossible or meaningless. A comprehensive explicit representation of all conceivable combinations of features is dispensed with here only for the sake of brevity and readability of the description.

Further features and possible combinations of features by way of example as well as the advantages linked to those features and combinations of features are described by reference to the following examples.

### EXAMPLES

The following examples describe point of care immediate diagnostics of blood clotting in the case of a polytraumatised patient in accordance with the present invention.

### Example 1

350 μl of blood is taken from a patient whose blood clotting activity is to be determined by means of thrombelastography. A gas-tight syringe is used for taking the blood sample, the syringe containing the smallest possible volume of an unbuffered trisodium citrate solution and a magnetic agitator. The blood sample is then under anaerobic conditions immediately after being taken. Blood and citrate solution are then mixed by repeatedly tilting the syringe. Thereafter the sample is transferred into the measurement chamber of the apparatus which is temperature-controlled to the patient temperature. In that case the sample is under a layer of a gas barrier fluid. Finally the ram rotation procedure is begun and the smallest possible volume of Ca²⁺ solution is added, in which case a time switch is triggered with the complete addition of the solution, by means of which time switch the timing of clot formation and fibrinolysis is determined in the context of the thrombelastography procedure carried out.

### Example 2

150 μl of blood is taken from a patient whose blood clotting activity is to be determined by means of determination of thrombocytes aggregation. A glass capillary which is coated on the inside with lithium heparinate is used for taking the blood sample under anaerobic conditions. Thereafter the sample is transferred into the measurement chamber of the apparatus which is temperature-controlled to the patient temperature, the measurement chamber being previously flushed with a water vapour-saturated gas mixture with a pCO₂ of 40 mmHg and pO₂ of 100 mmHg. During the subsequent operation of determining the blood clotting activity that gas mixture passes over the entire interface of the blood sample. As soon as the blood sample is completely transferred into the measurement chamber a time switch is triggered to determine the timing of impedance aggregometry, that is to say measurement of thrombocytes aggregation, using very small volumes of unbuffered reagents.

1. A method of extracorporeal determination of the blood clotting activity of a blood sample of an individual, in which the clotting time which elapses until the blood sample reaches a given degree of clotting or the degree of clotting reached by the blood sample within a given time is measured or in which in a corresponding manner the fibrinolysis time or the degree of fibrinolysis of a blood sample which has previously clotted to a certain degree is measured, wherein the pH-value is determined for a blood sample aliquot derived from the blood sample, measurement of the clotting/fibrinolysis time or the degree of clotting/fibrinolysis is effected with the exclusion of air and during the entire measurement of the clotting/fibrinolysis time or the degree of clotting/fibrinolysis the predetermined pH-value of the blood sample is maintained, which was determined for the derived blood sample aliquot.

2. A method according to claim 1 wherein the addition of additives to the blood sample is effected pH-neutral by the added substances either being so selected that they have no influence on the pH-value of the blood sample or in the case of a plurality of substances they are so selected that the influence of the individual substances on the pH-value of the blood sample is neutralised.

3. A method according to claim 1 wherein the volume of the total of the added reagents includes at most 5% of the volume of the blood sample in order not to influence the original pH-value of the sample by dilution.

4. A method according to claim 1 wherein all added reagents are unbuffered in order not to influence the original pH-value of the sample by buffer substances.

5. A method according to claim 1 wherein extraction of the blood sample from the body of the individual, storage of the blood sample until the measurement operation and transfer of the blood sample into an apparatus for measuring the clotting/fibrinolysis time and/or the degree of clotting/fibrinolysis are effected with the exclusion of air.

6. A method according to claim 1 wherein the blood sample is taken with a gas-tight syringe or with a glass capillary which is coated on the inside with lithium heparinate.

7. A method according to claim 1 wherein measurement is effected without contact of the blood sample with a gas phase or in an artificial atmosphere, wherein for example carbogen is used as the artificial atmosphere.

8. A method according to claim 1 wherein a barrier gas which is markedly heavier than air and lighter than blood such as for example sulphur hexafluoride (SF₆) is used to overlay the blood sample arranged in the measurement chamber so that the blood sample cannot come into contact with the gas phase above the barrier gas.

9. Apparatus for the extracorporeal determination of the blood clotting activity of a blood sample of an individual, wherein the apparatus has a measurement chamber for measuring the clotting time which elapses until the blood sample reaches a certain degree of clotting, the degree of clotting that the blood sample reaches within a given time, or in a corresponding fashion the fibrinolysis time or the degree of fibrinolysis of a blood sample previously clotted to a certain degree, wherein the measurement chamber is of such a configuration that measurement of the clotting/fibrinolysis time or the degree of clotting/fibrinolysis is effected with the exclusion of air, wherein the measurement chamber is of such a configuration that measurement is effected without contact of the blood sample with a gas phase, wherein the apparatus has means for determining the pH-value of the blood sample in the measurement chamber.

10. Apparatus according to claim 9 wherein the internal surfaces which come into contact with the blood plasma, in particular the measurement chamber and the feed lines which are possibly provided and by way of which the blood sample can be introduced into the measurement chamber have low gas absorption properties.

11. (canceled)

12. Apparatus according to claim 9 wherein it has a measurement chamber having a surface-volume ratio of the surface of the measurement chamber, that comes into contact with the blood sample, to the volume that a blood sample of a volume of 100 to 500 μl occupies in the measurement chamber in the range of 10:1 cm⁻¹ to 25:1 cm⁻¹.

13. Apparatus according to claim 9 wherein in the cases in which a gas phase is present in the measurement chamber during the measurement operation the ratio of the interface between the blood sample and gas phase with respect to the volume of the blood sample in the measurement chamber is to be such that the surface-volume ratio achieves at most a value of 1:1 cm⁻¹.

14. Apparatus according to claim 9 wherein the measurement chamber is of such a configuration that in the case of a measurement chamber which is completely filled during the measurement operation, of a volume of between 100 and 500 μl the ratio of the interface between the blood sample and the surface of the measurement chamber to the volume of the blood sample is in the range of 10:1 cm⁻¹ to 25:1 cm⁻¹.

15. An apparatus according to claim 9 wherein the clotting time to be determined is preferably selected from the thromboplastin time (TPT), the activated partial thromboplastin time (aPTT), the thrombin time (TT), the prothrombin time, the ecarin clotting time (ECT) and the activated clotting time (ACT) or that the method or the apparatus is used in thrombelastography, in impedance aggregometry or in the measurement of thrombocytes adhesion and aggregation.

16. A method-according to claim 1 wherein the clotting time to be determined is preferably selected from the thromboplastin time (TPT), the activated partial thromboplastin time (aPTT), the thrombin time (TT), the prothrombin time, the ecarin clotting time (ECT) and the activated clotting time (ACT) or that the method or the apparatus is used in thrombelastography, in impedance aggregometry or in the measurement of thrombocytes adhesion and aggregation.
